(19) United States
(12) Patent Application Publication
(10) Pub. No.: US 2007/0105210 A1
(43) Pub. Date: May 10, 2007

(54) Work device comprising bordered work zones, on-chip laboratory and microsystem

(75) Inventors: Cyril Delattre, Izeaux (FR); Gilles Marchand, Pierre Chatel (FR); Patrick Pouteau, Meylan (FR); Frederic Ginot, Saint-Egreve (FR)

Correspondence Address: MCKENNA LONG & ALDRIDGE LLP, 1900 K STREET, NW, WASHINGTON, DC, 20006, US

(73) Assignees: COMMISSARIAT A L'ENERGIE ATOMIQUE, Paris I 5eme (FR); BIOMERIEUX SA, Marcy L 'Etoile (FR)

(21) Appl. No.: 10/577,274

(22) PCT Filed: Oct. 21, 2004
(86) PCT No.: PCT/FR04/50525

§ 371 Date: Nov. 6, 2006

(30) Foreign Application Priority Data
Oct. 31, 2003 (FR) 03/50762

(51) Int. Cl. C12M 1/34; H01L 21/00

(52) U.S. Cl. 435/287.2; 438/1

(57) ABSTRACT

The present invention relates to a work device (1) comprising work zones. It can be used to obtain a matrix of drops on a surface, using a liquid of interest (E). It comprises a work box (Bo) provided with means (o, s) for introducing and extracting the liquid respectively into and from the box; a substrate (S) comprising an active surface that is substantially non-wetting for said liquid of interest contained in said box; distinct work zones (Zt) formed on said active surface and each surrounded by a border (b) formed on said active surface, the borders not touching one another and having no common edge and having a geometry such that when the liquid of interest is extracted from the box, a drop (g) of the liquid of interest remains imprisoned by each border and in contact with the work zone that it surrounds.

The instant application claims the priority of the French patent application filed on Oct. 31, 2003 under number 03 50762, which is incorporated herein by reference.

### TECHNICAL FIELD OF THE INVENTION

The present invention relates to a work device comprising bordered work zones, to an on-chip laboratory and to a microsystem comprising this device, particularly a biological chip. The present invention further relates to a method for producing a device of the invention.

The present invention makes it possible to obtain a high-density matrix of drops localized on a surface, from a liquid of interest. It enables the easy transition from a closed fluid chamber, called a work box, and filled with a liquid of interest, to a matrix of drops, or microvolumes, perfectly localized on a surface placed in said chamber, when the liquid of interest is removed from said fluid chamber.

The term matrix of drops means a predefined arrangement of said drops, without requiring any particular geometric shape of said arrangement. The matrix of drops may be round, square, polygonal and even random, the essential factor being that the drops formed are arranged in a localized and predefined manner on the surface according to the objective achieved by the present invention. Within the context of the present invention, the term “localized” means circumscribed, individualized and distinct from the other drops deliberately captured on said surface using the device of the invention.

Each of the drops may be subjected to one or more operations for qualitatively and/or quantitatively analysing one or more analytes present or likely to be present in the liquid of interest, for example, a molecule, an oligonucleotide, a protein, etc. The analytes in the drop can be analysed by any technique known to a person skilled in the art for performing the analyses, particularly in a liquid volume as small as a drop. It may involve analytical techniques used on biological chips. The analysis may or may not involve the surface of the device of the-invention covered by the drop, according to the implementation of the present invention.

Each of the drops forms a volume in which chemical or biochemical reactions can be carried out. Any chemical or biochemical reaction known to a person skilled in the art can be carried out in this volume. These reactions may or may not involve the surface of the device of the invention covered by the drop, depending on the implementation of the present invention. When these reactions involve the surface of the device of the invention covered by the drop, they may take place with a single drop or several drops deposited in succession on this surface, these successive drops consisting of a single or a plurality of different liquids of interest according to the implementation of the present invention. One example of chemical reactions involving two different liquids of interest on a device of the invention is the following: using a drop of a first liquid of interest, localized deposition of a film of an organic polymer on the surface covered by this drop, and then, using a drop of a second liquid of interest, functionalization of the organic polymer film deposited on this surface.

According to the present invention, chemical/biochemical analyses and reactions can be carried out exclusively on a device according to the present invention. In this case, they may be simultaneous (reaction and analysis) or successive (reaction followed by analysis or analysis followed by reaction). Furthermore, several analyses and/or several reactions may succeed one another. For example, the device of the present invention can advantageously be used, on the one hand, in the production of a card, or on-chip laboratory (for example by chemical reactions for depositing a polymer, followed by the functionalization thereof) (“lab-on-chip”), in which all the steps necessary for the qualitative and quantitative analyses of a liquid of interest are incorporated: handling of the fluid, chemical and/or biochemical reactions, optical, electrical and/or chemical detection chip, etc.; and on the other, in the use of this card, or on-chip laboratory, for carrying out qualitative and/or quantitative analyses in drops of a liquid of interest to be analysed (chemical/biochemical reaction(s) and analysis).

In the present description, the numerals in brackets [ ] refer to the appended list of references.

### PRIOR ART

Depending on the applications considered, this invention relates to the general field of the formation of drops, of work in microvolume(s), of matrices with a high drop density.

The formation of localized zones to isolate a liquid phase is commonly practised in the field of biological chips, particularly DNA chips. For these applications, the reaction volume is often very small to economize the biological products and reagents.

For the formation of localized drops and matrices with a high drop density, the companies Protogene Laboratories Inc. [1] and Affymetrix Inc. [2] use a technique employing an automated dispensing system. These systems lead to the formation of drops and of matrices with a high density of spots or drops on a surface.

However, besides the drop dispensing system, all these techniques require a device for the accurate movement and alignment of this system, as well as a liquid feed device. This apparatus is very costly. Furthermore, the maximum density-of the drop matrices which can be formed is limited by a combination between the size of the drops dispensed and the minimum inter-spot spacing of the dispensing system.

Two significant examples can be cited for the formation of matrices with a high density of micro-depressions: the formation of a network of depressions microfabricated by etching in a silicon wafer to obtain DNA amplicons by PCR in microvolumes of a few picolitres, and the formation of wells or channels by photolithography on photoresists deposited on a plastic substrate [3]. With these techniques, the number of wells varies from 100 to 9600 wells, with diameters of 60 to 500 μm and depths of 5 to 300 μm.

However, the edges of these depressions leave no physical separation between the liquid phase in the depression and that outside it, hence allowing connections between the depressions, and therefore contaminations between them. Furthermore, for their use, these devices require drop dispensing systems, a device for the accurate movement and alignment of the system, as well as a liquid feed device. This raises the same drawbacks and problems as those described above.

For electrical or electrochemical detection in biological tests, a large number of electrical or electrochemical detection systems described in the literature are unable to descend below nanomolarity in terms of detection limit, a limit that is often due to the small number of electrons generated by each hybrid.

Techniques involving enzyme accumulation help to lower this detection limit to about one picomole because of the high amplification of the number of redox species to be detected in the reaction medium [4]. However, this amplification method raises a problem for the multispot systems currently known, because the redox compound diffuses and may thereby contaminate the neighbouring spots.

For this purpose, most of the time, the use of three-dimensional structures (use of compartments) is recommended in the literature. For example, Infineon [6] proposes polymer walls and a system for molecule migration by electrical forces, in order to confine them in a defined volume and thereby prevent inter-spot contamination. Unfortunately, fluid filling problems may be encountered with this type of approach when, for example, working in a very fine liquid stream. Here also, a drop dispenser becomes indispensable.

Hence there is a real need for a device for easily obtaining a matrix of drops with a high density using a liquid of interest, usable without any drop dispensing apparatus, easy to produce, effectively avoiding contamination between drops, and which can be used very flexibly with all the methods currently known to a person skilled in the art for collectively or individually analysing microvolumes, for example, on an on-chip laboratory, whether for a chemical, electrical or optical process or a combination thereof.

### SUMMARY OF THE INVENTION

The present invention precisely meets this need, and others also explained below, by providing a work device comprising:
a work box provided with means for introducing a liquid of interest into the box and means for extracting the liquid of interest from the box,
a substrate comprising an active surface that is substantially non-wetting for said liquid of interest contained in said box,
a plurality of distinct work zones formed on said active surface and each surrounded by a border formed on said active surface that is substantially non-wetting for the liquid of interest, the borders not touching one another and having no common edge,

in which the means for introducing and extracting the liquid of interest respectively into and from the box are arranged on said work box in such a way that when the liquid of interest is introduced into the box, it covers the work zones and their respective border, and

in which the borders have a geometry such that when the liquid of interest is extracted from the box, after having been introduced therein, a drop of the liquid of interest remains imprisoned by each border and in contact with the work zone that it surrounds.

The present invention also meets this need by providing an on-chip laboratory comprising a device according to the invention.

The present invention also meets this need by providing a system comprising a device according to the invention.

The device of the present invention makes it possible, without a drop dispensing apparatus, to effect a transition of a volume of liquid of interest present in a fluid chamber, consisting of the work box, to a multitude of drops of said liquid that are retained by the independent micro-depressions created by the borders surrounding the work zones, in which, for example, an optical, electrical, magnetic, mechanical, electrostatic, etc., sensor or actuator may be present.

Within the context of the present invention, a liquid is said to be “of interest” if this liquid is intended to be captured by borders of a device according to the invention, so as to form a matrix of drops of this liquid.

The term “liquid of interest” means any liquid likely to require an arrangement as a matrix of drops on a support, for example for an analytical and/or chemical and/or biochemical purpose. The term “chemical and/or biochemical purpose” means any chemical and/or biochemical reaction that can be carried out in a liquid. The term “analytical purpose” means any qualitative and/or quantitative analysis that can be carried out in a liquid.

The liquid of interest may be organic or aqueous. It may be any one of the liquids currently handled in laboratories or in the industry, for example, in on-chip laboratories. It may, for example, be a liquid selected from a solution, a solvent, a reagent, a sample, a cell extract, a sampling taken from an animal or plant organism, a sampling taken from nature or industry, etc. It may be a biological or chemical liquid. This liquid of interest may be a liquid that is, if necessary, diluted for its use with the device of the present invention, as in the case of on-chip laboratories. A solid product can be placed in solution to create a liquid of interest within the context of the present invention. This solid product may be selected, for example, from a chemical or biochemical product, a reagent, a material to be analysed, a sampling taken from an animal or plant organism, a sampling taken from nature or industry, etc. A person skilled in the art knows the handling of such products and liquids of interest.

The substrate of the device of the invention actually constitutes the support on which the active surface is formed with its work zones and their respective border. The substrate may be made from any appropriate material for implementing the present invention. It may, for example, be one of the basic materials used to produce on-chip laboratories, biological chips, Microsystems, etc. It may, for example, be a material selected from the group consisting of silicon, silicon dioxide, silicon nitride, glass, plastic, an organic polymer, and a metal or a metal alloy. The organic polymers may, for example, be selected from the group comprising polycarbonates, polydimethylsiloxanes, polymethyl methacrylates, polychlorobiphenyls and cylcoolefin copolymers. The metal may be selected, for example, from the group consisting of Au, Ti, Pt, Al, Ni, Sn and the metal alloy may be stainless steel.

The term active surface means the surface of the substrate on which the work zones are formed surrounded by their border. According to the invention, the substrate may comprise one or more active surfaces. The active surface may consist of any material that is substantially non-wetting for the liquid of interest and suitable for implementing the present invention. In fact, the operation of the device of the present invention is partly based on the fact that the active surface retains very little or no liquid of interest, thereby permitting total and easy dewetting, without retention of the liquid of interest on the surface between the borders, and also without drying. Thus, the drops of liquid of interest are captured selectively and exclusively by the borders and are circumscribed to the work zones that they surround, thereby avoiding any problem of contamination between the drops, and hence between the work zones.

The term surface that is substantially non-wetting for the liquid of interest means a surface to which the liquid of interest has low adherence, that is, if the liquid of interest is made to flow on such a surface, it leaves no traces or drops. However, capture becomes difficult, indeed impossible, in the case in which the liquid of interest absolutely does not wet the surface. Similarly, if the surface is totally wetting, it becomes impossible to completely remove the liquid of interest. Thus, preferably, the substantially non-wetting surface makes a contact angle of at least 60°, preferably 60 to 90°, with the liquid of interest for which the device of the invention is intended. For example, if the liquid of interest is aqueous, the material forming the active surface is advantageously hydrophobic, preferably with a contact angle of 60 to 110°.

No chemical change is required in the substrate surface if the substrate consists of a material that is already substantially non-wetting for the liquid of interest.

In contrast, if the substrate surface is not already substantially non-wetting for the liquid of interest, a surface treatment may be necessary to make it substantially non-wetting. In this case, the material of the active surface is selected in particular according to the liquid of interest with which a matrix of drops is to be formed, according to the substrate, and also according to the work zones. It may be formed on the substrate by chemical modification of the substrate surface or by deposition on this surface of a material that is substantially non-wetting for the liquid of interest.

For example, if the liquid of interest is aqueous, the material forming the active surface is advantageously hydrophobic. For example, in the above examples of materials forming the substrate, the substrate surface can be made non-wetting, hydrophobic here, by chemical modification, for example by silanization with a silane containing hydrophobic functions, for example 1H, 1H, 2H, 2H-perfluorodecyltrichlorosilane. It may, for example, also be a deposit of liquid teflon on a turntable; a gas-phase silanization of hydrophobic silane; the use of a hydrocarbon silane, for example of the octadecyltrichlorosilane type. The materials and methods usable for implementing such chemical modifications are known to a person skilled in the art. One embodiment is described below.

The shape and size of this active surface, and hence also of the substrate on which it is formed, are irrelevant to the operation of the device of the invention. They may be determined, for example, according to the number of borders coupled to the work zones formed on the active surface, and, optionally, to their arrangement on this surface, and also according to the desired size of the device in its use configuration and to the cost specifications. To avoid undesirable retentions of the liquid of interest, between the borders, the substrate surface comprising the work zones and their border is preferably plane. By way of example, the active surface may have a shape and size comparable to those used in on-chip laboratories and analysis and detection microsystems known to a person skilled in the art.

According to the invention, the term “borders” means structures in relief formed on the substrate in order to create unjoined depressions. These depressions are not “embedded” in the substrate body, but are created on its surface by their border. FIG. 1 appended hereto shows a schematic representation in cross section of two types of depression: to the left, depressions (Cₐ) of the prior art, “embedded” in a substrate (Sₐ), and to the right, depressions (c) according to the present invention, that is, formed by their border (b) on a substrate (S). Hence a free space remains available between the borders of the depressions, according to the present invention, for flows of the liquid of interest. These borders hence each permit a highly localized capture of a drop (g) of the liquid of interest. The term “localized” is defined above. For example, in a basic use of the device, by making the liquid of interest flow on the active surface in order to cover these borders, and the depressions they form, the borders capture, or retain, a drop of liquid of interest in the depression, while the active surface, which is substantially non-wetting for the liquid of interest, retains very little or no liquid of interest. By stopping the flow of the liquid of interest, only one drop of this liquid is retained locally per border, on the work zone that it surrounds.

The exact shape of the borders, or wall, is not definitive and may be adapted according to the applications and means of production available for producing them. According to the invention, the borders may have any shape provided that they can, each, capture or imprison a drop of liquid of interest, and that this drop is in contact with the work zone surrounded by said border. By way of example, the borders may have a cross section in the direction from the active surface to the upper part of the border, selected from a triangular, rectangular, conical, frustoconical, semi circular, semi-elliptical shape. FIG. 2 schematically shows, in cross section, various possible geometries of borders (b) according to the invention formed on a substrate (S). Also by way of example, the borders may have a shape, around their work zone(s) and viewed from above, that is selected from an annular, star, rectangle, square, triangular, elliptical shape or a polygon having 4 to 20 sides. FIG. 3 is a schematic representation of borders (b) according to the invention, viewed from above, having various shapes around their work zone (Zt) which they surround.

The ratio of the height of the edges to the diameter of the depressions is a factor in controlling the proper retention of a drop of liquid of interest in the depressions. If the borders are too high for a given diameter, the liquid of interest cannot fill the depressions formed by these borders, and therefore cannot be retained. In contrast, if the height of the borders is too low for a given diameter, the liquid of interest is not retained in the depressions formed by these borders because they cannot play their role as an obstacle to suction. Thus, by way of example, according to the invention, the borders advantageously have the shape of a ring, optionally with one of the abovementioned geometric shapes, of which the height (h) above the active surface is 5 to 20 μm; where the cross section (e) of the ring at the level of the active surface is 20 to 100 μm; and where the diameter (D) inside the border, bounding the work zone, is 15 μm to 5 mm.

According to the invention, the active surface may also be defined as follows (see FIG. 1 for information for the numerals):
D: inside diameter of the drops, with, for example 15 μm≦D≦5 mm;
L: drop spacing;
e: widest section of the wall, with, for example, 20 μm≦e≦100 μm; and
h: wall height, with, for example, 5 μm≦h≦20 μm;

where h/D<0.15; e/D<0.33; and h/L<0.3.

The borders are made according to the following rules: they are wall-shaped relief structures defining a closed perimeter, with unjoined edges from one border to another. These borders may be produced by any method known to a person skilled in the art to shape the abovementioned materials of the substrate, or by any method known to a person skilled in the art to shape reliefs on a surface, particularly in the field of on-chip laboratories and analysis Microsystems, for example, by deposition of material(s) and etching. By way of example, among the methods known to a person skilled in the art usable to produce the borders according to the present invention, mention can be made of: direct etching of the substrate; deposition of a material on the surface of a plane substrate, for example by coating, evaporation, spraying or electroplating, followed by etching in combination with a conventional photolithography method, for example by coating with a resist, exposure and definition of features, or etching; direct definition of features by photolithography in photosensitive polymers, for example in the case of photoresists; moulding or stamping, for example of plastics or of the substrate forming the active surface.

The borders according to the invention can, in particular, be produced during the final step of a technological stacking of several layers on a substrate. The lower layers may contain actuators or mechanical, optical or electronic detectors, for example, of the MEMS or optical MEMS (“MicroElectroMechanical System”) type or grafted molecules of chemical or biological interest intended to form the work zones. The depressions may, for example, be arranged in a checkerboard pattern, on the substrate surface, so that they have no common edge.

According to the invention, optionally, the borders may be wetting for the liquid of interest on their uppermost part with respect to the active surface and/or on their slope opposite the work zone that it surrounds. This option makes it possible, if necessary, to reinforce the retention of the drop of liquid of interest captured by the border. This wettability can be obtained, for example, on borders consisting of silicon, silicon dioxide (SiO₂), glass, silicon nitride (Si₃N₄), that is, materials suitable for making the substrate, by grafting onto this material of a chemical function that is wetting for the liquid of interest, for which the device of the invention is intended. For example, the chemical function that is wetting for an aqueous liquid of interest can be selected from the group consisting of an alcohol, alcoholate, carboxylic acid, carboxylate, sulphonic acid, sulphonate, oxyamine, hydrazine, amine and ammonium function.

This wettability can also be obtained when the substrate is based on silicon by etching to form hydrophilic black oxidized silicon that requires no chemical modification to be wetting for aqueous solutions. This economical embodiment is thus preferably used when the liquid of interest is aqueous. Document [10] describes a protocol suitable for implementing this embodiment.

The type of the surface, outside of the depressions, and also advantageously inside the depressions, is an important parameter for the good overall functioning of the device of the present invention. The treatment of the substrate surface to make it substantially non-wetting can be effected before or after the formation of the depressions in order to modify the affinity of the zones on the substrate: between the depressions and, advantageously at their centre. Thus, the removal by suction of the liquid of interest is facilitated by a weak affinity between the liquid of interest and the surface between the depressions. Furthermore, the centre of the depressions may advantageously have a good affinity for the liquid phase to facilitate the capture of a drop of liquid of interest in the depressions.

Quite unexpectedly, the inventors observed that when the entire substrate surface, that is, the centre of the depressions formed by the borders and the surface between the depressions, has a poor affinity for the liquid of interest, particularly if it is substantially non-wetting for the liquid of interest, the capture of the liquid phase in the depressions during suction can nevertheless be effected due to the presence of the walls of the depressions according to the present invention, even if they are non-wetting for the liquid of interest. Thus, according to the invention, the work zones may be zones that are non-wetting for the liquid of interest. A further advantage of this invention is that the capture of the liquid of interest depends much less on the state of the surface or its change over time than for the devices of the prior art. In fact, if the affinity between the centre of the depression and the liquid of interest decreases over time, the capture remains ensured by the presence of the borders, or walls, according to the present invention.

Preferably, according to the invention, at least one work zone is in the same plane as the active surface, and more preferably all the work zones of the active surface. Since the borders are realised around the work zones, this accordingly facilitates the production of the device of the present invention.

Within the context of the present invention, the term work zone means a zone in which physical and/or chemical and/or optical operations can be performed in the drop captured by the border surrounding it (its border). Thus, according to the invention, at least one work zone may be an interaction zone selected from a zone of electrical, chemical, mechanical or optical interaction with said drop of liquid of interest captured, or a zone in which a plurality of these interactions are used simultaneously or successively.

Thus, according to a first embodiment of the invention, at least one work zone may be a zone of electrical interaction, for example, an electrochemical microcell. An electrochemical microcell is a device having at least two electrodes, preferably coplanar, forming a work electrode and a counter-electrode. It may also have a reference electrode. These elements are known to a person skilled in the art and the production methods known to a person skilled in the art can be used to produce this work zone, for example, the method described in reference document [7].

Thanks to this embodiment, the device of the present invention can constitute a genuine electrochemical microreactor that uses the drops of liquid of interest that are captured by the borders as reaction media and, more precisely, as electrochemical media. Each electrochemical reactor (border+work zone in the form of an electrochemical microcell+drop of liquid of interest captured) according to this first embodiment of the present invention can be used to perform any electrochemical reaction and/or analysis known to a person skilled in the art.

This reactor can serve, for example, to carry out localized electropolymerization reactions of one or more monomers present in the drop (polymerization or copolymerization) and/or localized electrografting reactions of one or more chemical molecules present in the drop of liquid of interest on one of the electrodes of the microcell. In this example, the liquid of interest may be a liquid containing the reagents necessary for the desired electropolymerization or electrografting. The polymerization and the grafting are then advantageously localized at the drop of liquid of interest captured by the border. Such localized electropolymerization or grafting reactions can be used, for example, to produce biological chips or analysis systems.

In a particular example, the electrochemical microcell of the device of the invention can be used first to “produce” the work zones, and then, for example, to use these work zones for the analysis of the drops of a liquid of interest to be analysed. For example, if the work zones are to comprise an organic polymer functionalized by a probe, for example a biological probe, they can be produced by electropolymerization of a conducting polymer functionalized by a probe, for example, following the method described in reference document [5]. The specificity of the use of the device of the invention is that the borders are used for the localized capture on each work zone of a first drop of a first liquid of interest containing the reagents necessary for electropolymerization (organic monomer). The functionalization by the probe can be achieved simultaneously with the electropolymerization, in which case the first liquid of interest also contains the probe (for example monomer functionalized by the probe). The functionalization can also be achieved subsequently to the electropolymerization by means of a second drop of a second liquid of interest (containing the probe) captured by the same borders and, accordingly, localized on the same work zones. Furthermore, the work zones thus produced can then be dried, and again, thanks to the border that surrounds them, can serve to capture a drop of a third liquid of interest to be analysed, containing a target that interacts with the probe (for example, complementary oligonucleotides). A fourth liquid of interest can also be used to analyse (detection and/or assay) the probe/target interaction on said work zones, and so on and so forth.

The electrochemical microreactor according to the invention can also serve, for example, to carry out electrochemical, qualitative and/or quantitative analyses of analytes present in the drops captured by the borders. It may also serve, for example, to carry out electrochemical, qualitative and/or quantitative analyses of a molecular probe/target interaction, the probe being fixed to the work zones, and the target being present in the drops of liquid of interest that are captured.

In one particular example, in which the electrochemical microcell of a device of the present invention is used to detect a target present in a liquid sample, for example by using an interaction of the target to be detected with a specific probe fixed to the work zones, it is possible to detect said interaction electrochemically, for example, by amplifying the signal by enzymatic accumulation in a drop of a liquid of interest, containing an enzymatic substrate, captured by the border surrounding each work zone. Document [4] describes an operational protocol usable for this type of detection, with the device of the present invention.

The detection of a probe/target interaction on a work zone may involve one of the means known to a person skilled in the art other than the electrochemical cell, for example, one of those discussed in the present description, for example, an optical method. The electrochemical microcell can therefore serve in this case exclusively to “produce” the work zones, the detection of a probe/target interaction then being effected by another means, or to analyse a probe/target interaction, the production of the work zones being carried out by another method, for example, one of those known to a person skilled in the art in the field of biological chips.

Irrespective of the implementation of this embodiment characterized by the presence of an electrochemical microcell, if a probe is used on the work zones, it can be selected, for example, from the group consisting of an enzyme, an enzyme substrate, an oligonucleotide, an oligonucleoside, a protein, a membrane receptor of a eukaryotic or prokaryotic cell, an antibody, an antigen, a hormone, a metabolite of a living organism, a toxin of a living organism, a polynucleotide, a polynucleoside, complementary DNA, or a mixture thereof. It is obviously selected according to the target with which it has to interact.

According to a second embodiment of the invention, at least one work zone may be a zone of chemical interaction with the drop of liquid of interest captured, without an electrochemical microcell. This work zone may, for example, comprise chemical or biological functions or reagents that are ready to react with a target of these functions or these reagents present in a liquid of interest. As for the first embodiment, the device of the invention can serve first to place these functions or these reagents on the work zones, and secondly, after drying, to capture a drop of liquid of interest containing the target of these functions or of these reagents.

This, at least one, work zone can be selected from those known to a person skilled in the art in the field of biological chips (chips sold by AGILENT, CIPHERGEN, EUROGENTEC). The difference between the device of the present invention and these chips of the prior art resides mainly in the presence of a border surrounding each work zone and permitting the capture of a drop of liquid of interest. This, at least one, work zone can be prepared, for example, by silanization followed by immobilization of biological probes, as described for example in the document referenced [8].

This, at least one, work zone can, for example, be a zone comprising a polymer functionalized by a biological probe like those mentioned above, with the purpose of fixing a corresponding target that may be present in a liquid of interest for detecting it, for example optically. For example, on a substrate like those mentioned above, this, at least one, work zone may be obtained by the methods described in the document referenced [9].

According to a third embodiment of the invention, at least one work zone may have active or measuring devices, such as sensors or actuators. This embodiment can be added to the abovementioned embodiments and variants, or be exclusive according to the goal to be achieved in the implementation of the present invention. The active or measuring devices are advantageously located at the centre of the surface of the work zones bounded by a border.

When a work zone comprises a sensor, it may be selected, for example, from the group consisting of electrical, magnetic, electrostatic, mechanical (for example pressure sensor), thermal (for example temperature sensors), optical (for example optical detection device) and chemical sensors.

When a work zone comprises an actuator, it may be selected, for example, from the group consisting of optical (light source), electrical, magnetic, electrostatic, mechanical (mechanical movement), thermal (heating resistor) and chemical actuators.

Such sensors and actuators, usable for the implementation of the present invention, and their method of production, are known to a person skilled in the art, particularly in the field of Microsystems. Here also, the difference between the device of the present invention and these chips of the prior art resides in particular in the presence of the border surrounding each work zone.

Irrespective of the embodiment of the method of the present invention, several work zones each surrounded by a border according to the invention can be arranged on a substrate. In a common application, for example, for the production of an on-chip laboratory, or of a microsystem, the number of work zones each surrounded by a border may, only for example, be 16 to 3025 per cm² of active surface. According to a variant of the present invention, several work zones may be surrounded by a single border, for example 2 to 4 or more, provided that when a drop of liquid of interest is captured by the border, this drop at least partially covers all the work zones which are surrounded by this border.

In general, thanks to the device of the present invention, various drops consisting of various liquids of interest may be captured successively by one and the same border and for different purposes, for example, to carry out the successive steps of a protocol for producing the work zone that it surrounds, for example, also to carry out the successive steps of detection and/or assay of an analyte in a liquid of interest. The advantage derived from the present invention is that irrespective of the objective of the successive captures of drops of liquids of interest, the drops successively captured are all localized on the work zones, thanks to their respective border.

The device of the invention also comprises a work box. This work box is a box used to cover the active surface of the device of the invention with the liquid of interest. This box can also be used to confine the active surface and/or to carry out analyses on or in the drops captured on the work zones. In the latter two cases, the device of the present invention accordingly constitutes a genuine miniature laboratory.

The device of the present invention may be used in Microsystems such as analytical Microsystems, or to form a biological chip, for example selected from the group consisting of nucleic acid, antibody, antigen, protein and cell chips.

The dimensions of this box depend in particular on the dimensions of the substrate provided with its active surface which must be introduced therein, but also, if applicable, other analytical devices or Microsystems which may be added to said box, for example, other on-chip laboratories. They may be lower than 1 cm for their largest side.

The box can, for example, be made from a material selected from the group consisting of an organic polymer, an elastomer, a glass, metal, silicon, a photoresist or any other material known to a person skilled in the art for implementing the present invention. For example, it may be a polymer selected from the group comprising polycarbonates, polydimethylsiloxanes, polymethyl methacrylates, polychlorobiphenyls and cycloolefin copolymers.

The material of the box is generally selected according to the type of liquid of interest to be introduced therein, the use of the box (simple covering of the substrate by the liquid of interest to form the drop matrix, or covering and analyses or other functions (chemical, electrochemical or biochemical reactions) and according to the manufacturer's cost specification. It may be made from material identical to the substrate of the device of the invention or different therefrom.

The box is preferably sufficiently sealed to avoid, for example, leaks during the introduction of the liquid of interest therein and/or contamination liable to enter from outside the box, for example bacterial, chemical, etc. and/or evaporation of drops captured by the borders surrounding the work zones of the device of the present invention.

According to a particular embodiment of the box, if the substrate and the box are made from the same material, the substrate may constitute one of the walls of the box, the active surface being directed towards the interior of the box.

The walls of the box may be assembled from, and on, the active surface of the device of the invention, for example by bonding or compression.

The work box may comprise a cover for its assembly, and also, in certain applications, for opening or closing it, particularly in order to remove therefrom the substrate of the invention with its active surface after having contacted it with the liquid of interest, or after the analyses or reactions in the drops. In fact, a single box can also serve to immerse, simultaneously or successively, one or, depending on its design, several substrates according to the invention. The box may then comprise removable means for fixing the substrate or substrates therein, for example, clips. If the box comprises a cover, it is preferably sufficiently sealed to avoid disturbing the introduction of the liquid of interest into the box.

The cover may be made from a material such as those mentioned above for the box. It may be produced, for example, by moulding, stamping, etching or mechanical erosion, etc. It may then be fixed definitively to the box to close it, for example by bonding, compression, plating or any other means known to a person skilled in the art for guaranteeing the integrity and leaktightness required for the use thereof. It may also be fixed removably to the box, while continuing to guarantee the integrity and leaktightness required for the use thereof, so that the same box thereby formed can serve to place matrices of drops on the several different substrates according to the invention, and/or with various liquids of interest.

Preferably, the material of the box and, if applicable, its cover, is, therein, substantially non-wetting for the liquid of interest. In fact, this serves to prevent drops from adhering to the inner surfaces of the box, after the extraction of the liquid of interest, and from falling on the active surface and hindering the analyses and reactions on the work zones in the drops captured by the borders. Surface treatments may be necessary to obtain this result, as for the active surface of the device of the invention. These treatments may, for example, be those described for the production of the active surface.

The box of the present invention may be provided with means for introducing the liquid of interest into said box and for extracting the liquid of interest from said box. These means may comprise, for example, two openings. There is no limitation on the position, shape, number and function of these openings other than the following: they must permit the introduction of the liquid of interest into the box followed by extraction of said liquid therefrom, and they must be arranged in such a way that when the liquid of interest is introduced into the box, it covers the border or borders of the active surface and when the liquid of interest is extracted from the box, one drop of liquid of interest remains captured per border. The liquid of interest can enter and exit the box via two different openings. It may also enter and then exit the box via only one of the two openings, a second opening serving for the extraction of the liquid of interest, either by allowing the passage of the air required for the extraction of the liquid of interest, or by injecting a gaseous fluid through this second opening in order to expel the liquid of interest from the box.

The means for introducing and extracting the liquid of interest respectively into and from the box comprise in particular openings which may be arranged on the cover or on the walls of the box, for example by etching, stamping, moulding, exposure to light in the case of a photoresist, mechanical drilling, etc.

The means for introducing the liquid of interest into the box may comprise any appropriate means known to a person skilled in the art for injecting a liquid into a box, particularly those used in the field of on-chip laboratories and Microsystems. These introduction means may be selected, for example, from a syringe, a pipette, a micropipette, an injection pump, etc.

The means for extracting the liquid of interest from the box may comprise any appropriate means known to a person skilled in the art for extracting a liquid from a box, particularly those used in the field of on-chip laboratories and Microsystems. These extraction means may, for example, be a manual or automatic extraction pump.

For example, according to the invention, if the means for extracting the liquid of interest comprise an extraction pump, this may be in the form of a pump for injecting a gaseous fluid into the box, via a first opening formed in the box, so as to be able to inject into the box a gaseous fluid expelling the liquid of interest from the box via a second opening formed on the box. Advantageously, the gaseous fluid injection pump further comprises a device for saturating the gaseous fluid injected with vapour of the liquid of interest. This saturation serves to prevent or limit the evaporation of the drop or drops captured by the borders.

Also for example, if the extraction means comprises a suction pump, this may be in the form of a suction pump arranged at one opening formed on the box so as to be able to extract the liquid of interest from the box by sucking it out via this opening. Advantageously, a second opening may be arranged on the box so as to permit the introduction of a gaseous fluid, for example air, an inert gas, or a gaseous fluid saturated with vapour of the liquid of interest, by the air intake caused by the suction of the liquid of interest.

The present invention further relates to a method for producing a device according to the invention, said method comprising the following steps:
supplying a substrate,
forming work zones on said substrate,
structuring the substrate surface so as to form a border around the work zones,
treating the surface on which the work zones and their border have been formed so as to make it substantially non-wetting for the liquid of interest,
supplying a box and introducing therein the substrate comprising the work zones surrounded by their border, said box comprising means for introducing the liquid of interest into the box and means for extracting the liquid of interest from the box, and
closing said box.

The substrate, the formation of the work zones, the structuring of the surface intended for forming the borders around the work zones, the treatment of the substrate surface for making it substantially non-wetting, are already defined above.

Within the context of the present description, it is clearly understood that the method of the invention includes the simultaneous or successive formation of several work zones and respective borders around them.

The various materials and steps of this method have already been described above.

The progress of the method permitting the capture of a drop of liquid of interest per depression formed by a border, on the active surface in the work box, can be described roughly as follows:
total or partial filling of the box, or fluid chamber, with the liquid of interest in order to cover the capture zone(s), then
extraction of the liquid of interest from the box.

Only the capture zone or zones each retain a drop of liquid of interest, the active surface being non-wetting. No further costly apparatus for dispensing drops is necessary. Moreover, the number of work zones is no longer limited by the limits of this apparatus.

The inventors of the present invention have also observed that, surprisingly, the extraction of the liquid of interest is effected more easily than on a substrate of the prior art, in which the edges of the depressions—which are not really depressions because the surface of these substrates is hollowed to form the depressions, but no border is formed in the sense of the present invention—are joined, because the zones released between the depressions form as many channels for the flow of a fluid. Furthermore, the particular arrangement of the work zones and borders of the present invention, combined with the production in relief on the surface, serves to prevent any communication of liquid from one depression to another, once suction has been effected.

The use of the device of the present invention is highly flexible, because it is possible to conduct in succession an operation that takes place collectively, followed by individual operations at each of the drops formed. Thus, in a first operation, called collective operation, the device of the invention permits the passage of a fluid stream of liquid of interest, for example, injected into said box, to a matrix of drops, or microvolumes, independent from one another. Then, the detection methods and/or chemical or biochemical reactions known to a person skilled in the art can be implemented individually (individual operation), in parallel, or successively, in each of the drops captured by the borders to detect and analyse the targets present in the liquid of interest.

In multi-step methods using the device of the invention, it is not necessary for all the steps to lead to the formation of drops. In fact, certain steps can perfectly well be carried out by covering all the borders with the liquid and then draining the box of this liquid so that no drops captured by the borders remain, for example by injection of a pressurized gas into the box, by vigorous agitation, etc.

It is moreover possible to capture various drops of one or more liquids of interest successively on one and the same work zone thanks to the border surrounding it. Each liquid of interest may contain one or more reagents necessary, for example, to carry out one of the steps of a chemical or biochemical method, for example to fabricate the work zones and/or to perform analyses. The succession of the various drops on one and the same work zone makes it possible, for example, to carry out various successive steps of a method implemented on the device of the invention, and, more particularly on the work zones surrounded by their border. All these method steps are thus advantageously localized on the work zones thanks to their border.

In experiments associated with the implementation of the present invention, the inventors found that the device of the invention solves other technical problems, compared with the techniques of the prior art, in the fields of on-chip laboratories, biological chips and Microsystems. In particular, the prior art describes a number of methods for localized covalent grafting of biological molecules in order to functionalize surfaces of the biological chip. This localization is generally carried out by a chemical, photochemical or electrical method. By a chemical method, the immobilization of biological element (probe) is effected by localized deposition (“spotting”) or in situ synthesis, which imposes constraints in terms of time. By photochemical analysis, it is possible to synthesize oligonucleotides using photolabile groups [4]: here also, limitations in terms of synthesis time and volumes of costly reagents are often encountered. Moreover, non-selective free-radical reactions may also take place. By the electrical method, the synthesis of oligonucleotides on solid support with electro-labile group faces the same limitations. By the electrochemical method [3], by copolymerization of pyrrole and of pyrrole bearing a biological species on a metal electrode. The latter technique has the drawback of requiring large volumes of costly reagents (pyrrole bearing the biological species).

The device of the present invention serves to solve these numerous problems of the prior art. In fact, it serves to rapidly and accurately functionalize surfaces of biological chips, which have become the work zones in the present invention, thanks to a rapid and accurate localization of each drop of liquid of interest on the work zone or zones, and accurate control of the densities of the immobilized probes. Furthermore, compared with the prior art methods, the volumes of reagents used are much smaller because of the accurate localization of the reaction in the volume of the drops of reagents captured by the capture zones. Moreover, the inventors' experiments have shown that the device of the present invention serves to work with microvolumes that are independent of one another, without cross-contamination between the detection spots, thereby considerably increasing the accuracy and reproducibility of the analyses.

Thus, the present invention permits, inter alia, an electrochemical or optical measurement in a confined environment, in the drops captured by the borders, and also a localized functionalization on the work zone by an electrochemical or chemical method with costly reagents: the volume of the reagents is reduced to the actual useful zone formed by each work zone surrounded by its border according to the invention.

This invention currently finds its greatest advantage in the on-chip laboratory and microsystem applications. The present invention hence also relates to a biological chip, for example, selected from the group consisting of nucleic acid, antibody, antigen, protein and cell chips.

According to the invention, detections of the various molecules that may be present in the liquid of interest can be carried out in parallel, simultaneously or successively, in various drops of liquid of interest captured on said active surface in the box.

According to the invention, the, at least one, analyte to be detected may be selected, for example, from biological or chemical molecules. The biological molecules may be selected, for example, from the group consisting of an enzyme, an enzyme substrate, an oligonucleotide, an oligonucleoside, a protein, a membrane receptor of a eukaryotic or prokaryotic cell, a virus, an antibody, an antigen, a hormone, a metabolite of a living organism, a toxin of a living organism, a nucleotide, a nucdeoside and a complementary DNA. The chemical molecule may be any molecule that must be analysed qualitatively and/or quantitatively.

Other features and advantages will further become apparent to a person skilled in the art from reading the examples below, provided for illustration and without being limiting, with reference to the figures appended hereto.

### BRIEF DESCRIPTION OF THE FIGURES

FIG. 1 is a schematic representation in cross section of two types of depression: to the left, depressions of the prior art, and to the right, the depressions according to the present invention.

FIG. 2 is schematic representation in cross section of various geometric shapes of borders according to the present invention.

FIG. 3 is a schematic representation of borders according to the invention, in plan views, having various shapes around the respective work zones that they surround.

FIG. 4 is a diagram showing a cross section of a device according to the present invention and its operation for the creation of a matrix of drops thanks to the active surface of its substrate.

FIG. 5 is a diagram showing a cross section of a device according to the present invention in which the means for introducing the liquid of interest into the box and for extracting the liquid of interest from the box use the same opening of the box.

FIG. 6 is a plan view, made from experimental photographs, of an active surface according to the invention, showing the formation of a matrix of drops: to the left, the surface without drops before the liquid of interest is introduced into the device of the present invention, and to the right, the surface with the matrix of drops retained by the borders (b) when the liquid of interest has been extracted from the box.

FIG. 7 is a photograph of an embodiment of the device of the invention in which a resin border surrounds each work zone, and in which the work zones are electrochemical microcells. The outside diameter of the resin ring surrounding the electrochemical cell of the device photographed is actually 700 μm.

FIG. 8 is a graph of curves of cyclic voltammetry measuring the current (I(μA)) as a function of the voltage (mV) before (Av) the formation of a matrix of drops and after (Ap) the formation of a matrix of drops on a device according to the present invention, whereof the active surface is shown enlarged in FIG. 7.

FIG. 9 is a schematic representation of various possible embodiments of a work box according to the invention, and in particular, it shows examples of arrangements of the means for introducing and for extracting the liquid of interest respectively into and from the box on various work boxes according to the present invention.

### EXAMPLES

### Example 1

### Production of Capture Zones Formed from Borders

A photolithography step is carried out on a fresh silicon wafer with a Clariant AZ4562 (trade name) thick photoresist as follows:
deposition of an adhesion promoter, which is hexamethylenedisilazane here, in an oven at 120° C.,
spin-coating of resin at 1000 rpm for 30 seconds with an acceleration of 200 rpm/s,
annealing on hotplate at 115° C. for 2 minutes,
insolation on Karl Süss MA750 (trade name) exposure machine for 50 seconds in batch mode (5×10 seconds with 5 seconds pause) through a mask,
development in a Shipley MF319 (trade name) solution diluted in proportions of 1:3 with deionized water,
cleaning with deionized water and drying under nitrogen stream,
annealing on a hotplate at 115° C. for 3 minutes, then at 150° C. for 1 minute,
thickness measurement: 13 μm.

On the mask used for insolation, all the motifs represent rings of which the walls have a width of 35 μm with various combinations between their diameter (100 to 1000 μm) and the inter-centre distance between two rings (50 to 1000 μm).

3025 depressions on a surface of one square centimetre were easily obtained.

### Example 2

### Production of the Box

A hollow cover of polydimethylsiloxane (PDMS) is produced by moulding on a glass mould with a square pattern and an oventhickness of 1 mm. On a plane device like those obtained with the preceding example, this hollow cover is fixed hermetically by bonding with crosslinking adhesive by irradiation with ultraviolet radiation (VITRALIT 6181). The connections for the fluid inlets and outlets are made by drilling the cover with small-diameter needles. The inlet needle is connected to fluid transport tubes and to a syringe filled with liquid of interest. The final assembly is tested for leaks, in the knowledge that the liquid must only pass through the connections provided for this purpose.

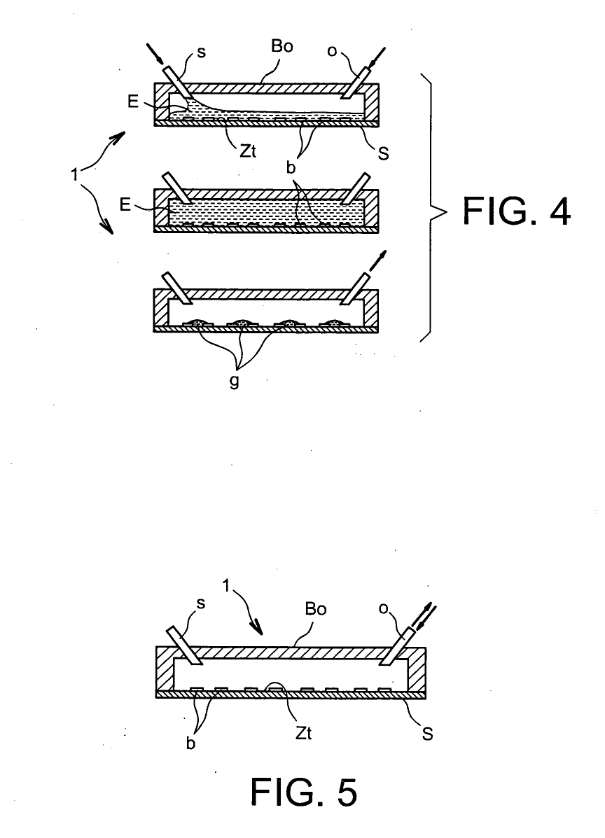

FIG. 4 is a schematic representation of the box obtained in this example. Other arrangements of the inlet and outlet connections (o, s) for introducing and extracting a liquid of interest can easily be obtained according to this example, and FIG. 9 schematically shows boxes that can be obtained.

In FIG. 4 appended hereto, the box (B) according to the invention comprises openings (o, s) The work zones (Zt) and borders (b) are also shown.

### Example 3

### Capture of Deionized Water on a Silicon Surface with Native Oxide

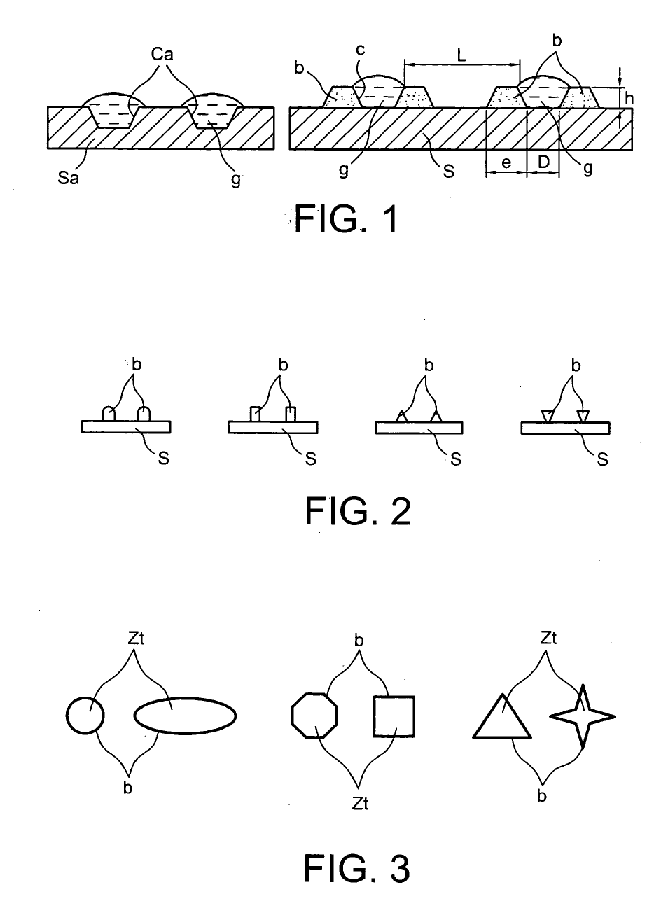

Various types of features forming borders according to the invention, shown in FIGS. 1 to 3 appended hereto, and obtained by the method described in example 1, are tested with deionized water (DW).

For this purpose, covers with a fluid stream about 1 mm thick created thanks to a work box according to the invention, produced according to example 2 (FIG. 4 appendedhereto) are used for the injection and extraction of DW, via plastic tubes.

The initial surface, consisting of silicon with a coat of native oxide, was not treated and the contact angle was close to 68° with the DW.

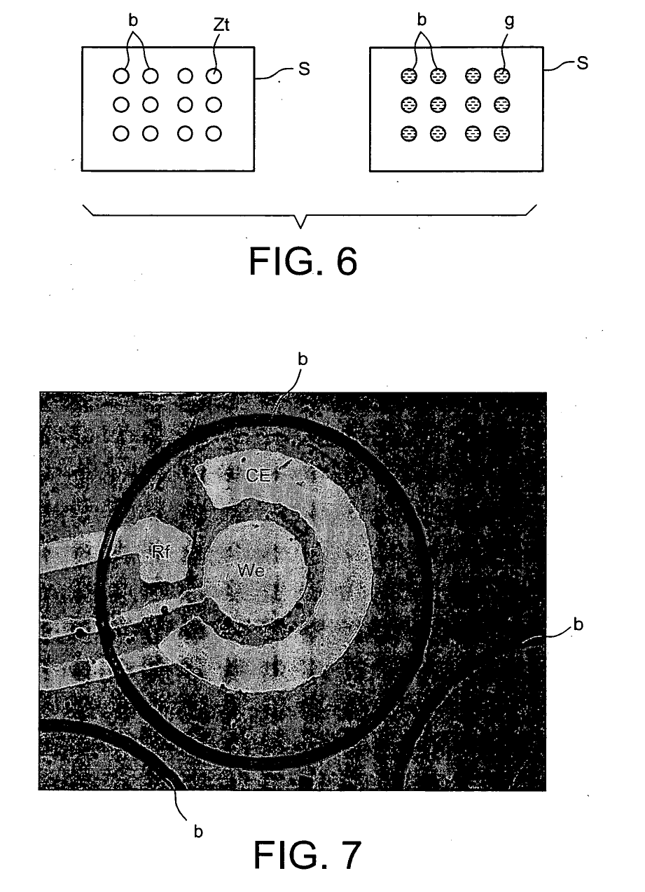

As shown in FIG. 6 appended hereto, to the right, the DW remains retained in the depressions formed by the border (b), on the work zone (Zt), in the form of drops (g) after removal of the liquid of interest by suction.

Various methods for filling the box with the liquid of interest were tested: with the introduction and extraction of the liquid of interest via the same opening (FIG. 5 appended hereto), and introduction via one opening and extraction via another opening (FIG. 4). A matrix of drops was obtained each time.

Furthermore, it was found that the filling of the box was not absolutely necessary, the important factor being that the borders are covered by the liquid of interest.

This example shows that a matrix of drops (g) properly localized on the work zones is obtained thanks to this device according to the present invention. Thus the present invention meets all the above-mentioned requirements of the prior art.

### Example 4

### Production of Work Zones Functionalized by a Probe According to the Invention

A technological stack using routine microelectronics techniques serves to form electrodes on a silicon wafer by metal deposition followed by photolithography followed by localized etching.

In this example, a microcell comprising three electrodes is produced and used. On an Si substrate with a 300 nm thick Si layer, the following steps, standard for a person skilled in the art of microelectronics, are carried out:
deposition of 300 nm of platinum (Pt) by sputtering;
photolithography in a photoresist with opening of the feature of the microcell and of the current inlet bands;
in a plasma reactor, complete ion etching of the Pt in the resist-free zones;
removal of the resist in a nitric acid bath;
in a plasma reactor, chemical vapour deposition of 500 nm of SiO₂;
photolithography in a photoresist with opening of the features of the electrodes of the microcell;
in a plasma reactor, complete ion etching of 500 nm of SiO₂ in the resist-free zones; and
removal of the resist in a nitric acid bath.

The working electrode (We) and the counter-electrode (CE) are made from platinum (deposit about 5000 Å) (see FIG. 7).

An Ag/AgCl/Cl⁻ reference electrode (Rf) is also present. This electrode is obtained by depositing silver on the platinum using the following protocol:
preparation of 10 ml of solution containing 0.2 M AgNO₃, 2 M KI, 0.5 mM Na₂S₂O₃,
potential of −0.65 V vs SCE (saturated calomel electrode) imposed for 90 seconds (monitoring by chronoammetry) on the reference electrode. A grey/white deposit is obtained. The substrate is then rinsed with water,
the substrate with the previously modified electrode is immersed in a 0.1M HCl solution and a potential of 0.5 V vs SCE is imposed for 30 seconds to chlorinate the silver deposit. The substrate is then rinsed with water.

A photolithography step identical to the one described in example 1 is then carried out in order to surround these previously obtained electrodes with a pad of Clariant AZ4562 (trade name) thick resist and to create depressions 500 μm in diameter with walls 13 μm high and 25 μm wide.

One of the borders (b) obtained is shown in full in the photograph in FIG. 7 appended hereto. It clearly surrounds the electrochemical microcell (CE, We, Rf).

The substrate is finally silanized with a hydrophobic silane (octadecyltrichlorosilane) according to the following method: the substrate is first treated to generate silanol sites in a Plassys MDS 150 (trade name) plasma reactor (Société Plassys, France) under the following conditions: power 400 W, reaction time 2 minutes, pressure 21.33 Pa (160 mTorr), oxygen flow rate 25 cm³/min., at ambient temperature. The substrate is then placed for 10 minutes at ambient temperature in a mixture of anhydrous heptane and hydrophobic silane with a silane concentration of 9 mM. It is then washed with heptane, then with toluene, then with water. The substrate is then placed in an oven for one hour at 110° C. The contact angle measured with water is close to 100°.

### Example 5

### Use of a Device According to the Invention for an Electrochemical Measurement with an Fe²⁺ Solution

The electrochemical cell surrounded by its border obtained in example 4 is tested using the box produced in example 2, and a solution containing ferrous (Fe II) ions is introduced into the fluid stream formed by this box.

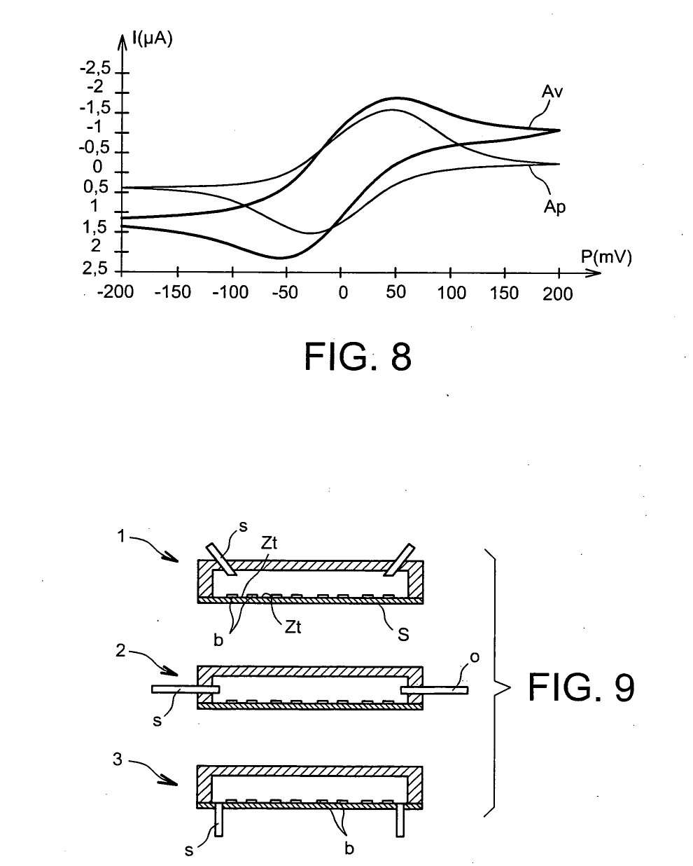

FIG. 8 is a graph of cyclic voltammetry curves measuring the current (I(μA)) as a function of voltage (mV) before the formation of the matrix of drops and after the formation of the matrix of drops on, a device according to the present invention shown in FIG. 7.

A first measurement is taken by cyclic voltammetry, showing the oxidation wave of the ferrous ions. The solution is then sucked out of the box to leave only the depressions each filled with one drop of liquid of interest. A second electrochemical measurement, identical to the first, is carried out, again showing the presence of the iron oxidation reaction.

The liquid of interest is hence clearly retained in the depressions, allowing a measurement after suction and drainage of the fluid chamber formed by the work box of the present invention.

The filling of the box is not absolutely necessary, the essential fact being that the borders are covered with the liquid of interest.

This example shows that a matrix of drops (g) clearly localized on the work zones is obtained thanks to this device according to the present invention. Hence the present invention meets all the above-mentioned requirements of the prior art.

### LIST OF REFERENCES

[1] WO 02/16023: Protogene Laboratories Inc.
[2] U.S. Pat. No. 6,040,193: Affymetrix Inc.
[3] WO 99/03684 : Eapen Saji et al.
[4] Azek et al., Analytical Biochemistry, 2000, 284, 107-113.
[5] WO 00/36145: Commissariat à l'Energie Atomique.
[6] WO 02/090573: Infineon
[7] J. Cooper et al., “Micromachining Sensor for Electrochemical Measurement in Subnanoliter Volumes”, Anal. Chem. 1997, 69, 253-258.
[8] FR-A-2 818 662
[9] EP 561 722
[10] H. Jansen et al., “The black silicon method: a universal method for determining the parameter setting of a fluorine-based reactive ion etcher in, deep silicon trench etching with profile control”, J. Micromech. Microeng. 5 (1995), 115-120.

1. Work device (1) comprising:
a work box (Bo) provided with means (o, s) for introducing a liquid of interest (E) into the box and for extracting the liquid of interest from the box,
a substrate (S) comprising an active surface that is substantially non-wetting for said liquid of interest contained in said box,
a plurality of distinct work zones (Zt) formed on said active surface and each surrounded by a border (b) formed on said active surface that is substantially non-wetting for the liquid of interest, the borders not touching one another and having no common edge,
in which the means (o, s) for introducing and extracting the liquid of interest respectively into and from the box are arranged on said work box in such a way that when the liquid of interest is introduced into the box (BO), it covers the work zones and their respective border, and
in which the borders have a geometry such that when the liquid of interest is extracted from the box, after having been introduced therein, a drop (g) of the liquid of interest (E) remains imprisoned by each border (b) and in contact with the work zone (Zt) that it surrounds.

2. Device according to claim 1, in which the, at least one, work zone is in the same plane as the active surface.

3. Device according to claim 1, in which the, at least one, work zone is a zone of electrical and/or chemical interaction with the drop captured by its border.

4. Device according to claim 3, in which the, at least one, work zone is an electrochemical microcell.

5. Device according to claim 1, in which the, at least one, work zone is a sensor selected from the group consisting of an optical, electrical, magnetic, electrostatic, mechanical, thermal or chemical sensor.

6. Device according to claim 1, in which the, at least one, work zone is an actuator selected from the group consisting of an optical, electrical, magnetic, electrostatic, mechanical, thermal or chemical actuator.

7. Device according to claim 1, in which the, at least one, work zone is a zone for detecting at least one chemical or biological species that may be present in the drop of liquid of interest when it is captured.

8. Device according to claim 7, in which the, at least one, work zone is a zone functionalized by a probe for interacting with a target that may be present in the drop of liquid of interest when it is captured.

9. Device according to claim 8, in which the probe is selected from the group consisting of an enzyme, an enzyme substrate, an oligonucleotide, an oligonucleoside, a protein, a membrane receptor of a eukaryotic or prokaryotic cell, an antibody, an antigen, a hormone, a metabolite of a living organism, a toxin of a living organism, a polynucleotide, a polynucleoside and a complementary DNA.

10. Device according to claim 1, in which the, at least one, work zone is a zone that is non-wetting for the liquid of interest.

11. Device according to claim 1, in which the substrate consists of a material selected from the group consisting of silicon, silicon dioxide, silicon nitride, glass, an organic polymer, plastic, tin, and a metal.

12. Device according to claim 11, in which the organic polymer is selected from the group comprising polycarbonates, polydimethylsiloxanes, polymethyl methacrylates, polychlorobiphenyls and cycloolefin copolymers.

13. Device according to claim 11, in which the metal is selected from the group consisting of Au, Ti, Pt, Al, Ni, and the metal alloy is stainless steel.

14. Device according to claim 1, in which the borders have a shape around the work zone and viewed from above, that is selected from an annular, a star, a rectangle, a square, a triangular, an elliptical shape, or a polygon having 4 to 20 sides.

15. Device according to claim 1, in which the borders have a cross section, in the direction from the active surface to the upper part of the border, selected from a triangular, rectangular, conical, frustoconical, semi circular or semi-elliptical shape.

16. Work device according to claim 1, in which the borders are wetting for the liquid of interest on their uppermost part with respect to the active surface and/or on their slope opposite the work zone that it surrounds.

17. Device according to claim 1, in which the borders are obtained by stamping or moulding of the active surface.

18. Work device according to claim 1, in which the means for introducing the liquid of interest into the box comprise a pump for injecting the liquid of interest into the box.

19. Work device according claim 1, in which the means for extracting the liquid of interest from the box comprise a pump for extracting the liquid of interest from the box.

20. Device according to claim 19, in which the pump for extracting the liquid of interest from the box is in the form of a pump for injecting a gaseous fluid into the box, via a first opening formed in the box, so as to be able to inject into the box a gaseous fluid expelling the liquid of interest from the box via a second opening formed in the box.

21. Device according to claim 20, in which the gaseous fluid injection pump comprises a device for saturating the gaseous fluid injected with vapour of the liquid of interest.

22. Device according to claim 19, in which the pump for extracting the liquid of interest from the box is in the form of a suction pump arranged at one opening formed in the box so as to be able to extract the liquid of interest from the box by sucking it out via this opening.

23. System comprising a device according to claim 1.

24. Biological chip comprising a device according to claim 1.

25. Biological chip according to claim 24, said chip being selected from the group consisting of nucleic acid chips, antibody chips, antigen chips, protein chips and cell chips.

26. Method for producing a device according to claim 1, said method comprising the following steps:
supplying a substrate,
forming work zones on said substrate,
structuring the substrate surface so as to form a border around the work zones,
treating the surface on which the work zones and their border have been formed so as to make it substantially non-wetting for the liquid of interest,
supplying a box and introducing therein the substrate comprising the work zones surrounded by their border, said box comprising means for introducing the liquid of interest into the box and means for extracting the liquid of interest from the box, and
closing said box.

27. Production method according to claim 26, in which the borders are formed on the active surface by direct etching of said active surface.

28. Production method according to claim 26, in which the borders are formed on the active surface by deposition of a material on said active surface followed by etching or photolithography of said material.

29. Production method according to claim 28, in which the material deposited is selected from the group consisting of a resin, a photoresist, organic polymers, metals, Si, oxidized Si, and Si nitride.

30. Production method according to claim 28, in which the deposition of a material on said active surface to form the borders is carried out using a method selected from coating, evaporation, spraying and electroplating.

31. Production method according to claim 28, in which, the material being photosensitive, the borders are made by photolithography.

32. Production method according to claim 26, in which the borders are obtained by stamping or moulding of the active surface.
